(19) United States
(12) Patent Application Publication
(10) Pub. No.: US 2004/0241089 A1
(43) Pub. Date: Dec. 2, 2004

(54) Systems and methods for detecting vulnerable plaque

(75) Inventors: Mark Brister, Forestville, CA (US); Robert Cafferata, Santa Rose, CA (US); Wenda Carlyle, Silverado, CA (US); Patrice Tremble, Santa Rose, CA (US)

Correspondence Address: Medtronic Vascular INC, 3576 Unocal Place, Santa Rosa, CA, 95403, US

(73) Assignee: Medtronic Vascular, Inc., Santa Rosa, CA (US)

(21) Appl. No.: 10/827,811

(22) Filed: Apr. 20, 2004

Related U.S. Application Data
(60) Provisional application No. 60/465,158, filed on Apr. 24, 2003.

(51) Int. Cl. A61K 51/00; A61K 49/00

(52) U.S. Cl. 424/1.11; 424/9.3; 424/9.6

(57) ABSTRACT

The invention provides systems and methods for detecting a vulnerable plaque associated with a blood vessel of a patient. A first aspect of the invention includes a substance that is administered to the patient and a device that detects the substance. The substance has affinity for/binds to at least one of a lipid, a clotting factor, or an apoptotic factor associated with the vulnerable plaque. A second aspect includes a particle that is administered to the patient, an emitter that emits infra-red or near infra-red radiation on the particle, and a detector that detects light fluorescence from the particle. A third aspect includes a substance that is administered to the patient. The substance has affinity for/binds to the vulnerable plaque and includes a substance radiopaque characteristic that activates upon association/binding of substance with the vulnerable plaque. A device that detects the substance radiopaque characteristic is also provided.

### RELATED APPLICATION

This application claims priority to U.S. Provisional Application No. 60/465,158, “Systems and Methods for Detecting Vulnerable Plaques” to Mark Brister et. Al., filed Apr. 24, 2003, the entirety of which is incorporated by reference.

### TECHNICAL FIELD OF THE INVENTION

The present invention relates generally to the field of vascular therapies. More particularly, the invention relates to systems and methods for detecting a vulnerable plaque associated with a blood vessel of a patient.

### BACKGROUND OF THE INVENTION

Heart disease, specifically coronary artery disease, is a major cause of death, disability, and healthcare expense. Until recently, most heart disease was considered to be primarily the result of a progressive increase of hard plaque in the coronary arteries. This atherosclerotic disease process of hard plaques leads to a critical narrowing (stenosis) of the affected coronary artery and produces anginal syndromes, known commonly as chest pain. The progression of the narrowing reduces blood flow, triggering the formation of a blood clot. The clot may choke off the flow of oxygen rich blood (ischemia) to heart muscles, causing a heart attack. Alternatively, the clot may break off and lodge in another organ vessel such as the brain resulting in a thrombotic stroke.

Within the past decade, evidence has emerged changing the paradigm of atherosclerosis, coronary artery disease, and heart attacks. While the build up of hard plaque may produce angina and severe ischemia in the coronary arteries, new clinical data now suggests that the rupture of sometimes non-occlusive, vulnerable plaques causes the vast majority of heart attacks. The rate is estimated as high as 60-80 percent. In many instances vulnerable plaques do not impinge on the vessel lumen, rather, much like an abscess they are ingrained under the arterial wall. For this reason, conventional angiography or fluoroscopy techniques are unlikely to detect the vulnerable plaque. Due to the difficulty associated with their detection and because angina is not typically produced, vulnerable plaques may be more dangerous than other plaques that cause pain.

The majority of vulnerable plaques include a lipid pool, necrotic smooth muscle (endothelial) cells, and a dense infiltrate of macrophages contained by a thin fibrous cap some of which are only two micrometers thick or less. The lipid pool is believed to be formed as a result of a pathological process involving low density lipoprotein (LDL), macrophages and the inflammatory process. The macrophages oxidize the LDL producing foam cells. The macrophages, foam cells, and associated endothelial cells release various substances, such as tumor necrosis factor, tissue factor and matrix proteinases, which result in generalized cell necrosis and apoptosis, pro-coagulation and weakening of the fibrous cap. The inflammation process may weaken the fibrous cap to the extent that sufficient mechanical stress, such as that produced by increased blood pressure, may result in rupture. The lipid core and other contents of the vulnerable plaque (emboli) may then spill into the blood stream thereby initiating a clotting cascade. The cascade produces a blood clot (thrombosis) that potentially results in a heart attack and/or stroke. The process is exacerbated due to the release of collagen and other plaque components (e.g., tissue factor), which enhance clotting upon their release.

Several strategies have been developed for the detection (e.g., diagnosis and localization) of vulnerable plaques. One strategy involves the measurement of temperature within a blood vessel. A localized increase in temperature is generally associated with the vulnerable plaque because of the tissue damage and inflammation. It has been observed that the inflamed necrotic core of the vulnerable plaque maintains a temperature of one or more degrees Celsius higher than that of the surrounding tissue. For example, a relatively normal vessel temperature may be about 37° C. whereas the vulnerable plaque may have a localized temperature as high as 40° C. Measurement of these temperature differences within the blood vessel may provide means for detecting vulnerable plaque. Alternatively, numerous other physical properties, changes, factors, molecules, and the like specific to the vulnerable plaque may allow detection. As such, it may be desirable to utilize anomalies specific to the vulnerable plaque to facilitate its detection.

Another strategy developed for the detection of vulnerable plaque involves the use of radioactive tracers. An example of such a strategy is disclosed in U.S. Pat. No. 6,295,680 issued to Wahl et al. According to the Wahl Patent, an intravenous solution containing a radioactive tracer, which specifically accumulates in the vulnerable plaque, is administered to the patient. A miniaturized radiation is positioned within the patient's arterial lumen (e.g., endovascularly) for localized radioactivity imaging and detection. The radiation detector identifies and differentiates vulnerable plaque from inactive, stable plaque. Although this strategy provides means for detecting and discriminating vulnerable plaque, the use of radioactive tracers may have disadvantages. For example, radioactive materials may have unwanted side-effects and typically require cumbersome handling and disposal procedures. Accordingly, it would be desirable to provide a strategy for detecting vulnerable plaque that does necessitate radioactive tracers.

Accordingly, it would be desirable to provide a strategy for treating vulnerable plaque that would overcome the aforementioned and other disadvantages.

### SUMMARY OF THE INVENTION

A first aspect of the invention provides a system for detecting a vulnerable plaque associated with a blood vessel of a patient. The system includes a substance that is administered to the patient and a device that detects the substance. The substance has affinity for at least one of a lipid, a clotting factor, or an apoptotic factor associated with the vulnerable plaque.

A second aspect provides a system including a particle that is administered to the patient, an emitter that emits infra-red or near infra-red radiation on the particle, and a detector that detects light fluorescence from the particle.

A third aspect provides a system including a substance that is administered to the patient. The substance has affinity for the vulnerable plaque and includes a substance radiopaque characteristic that activates upon association of substance with the vulnerable plaque. The third aspect includes a device that detects the substance radiopaque characteristic.

A fourth aspect provides a method including administering a substance to the patient, binding the substance to at least one of a lipid, a platelet, or an apoptotic factor associated with the vulnerable plaque, and detecting the bound substance.

A fifth aspect provides a method including administering a particle to the patient, emitting infra-red or near infra-red radiation on the particle, and detecting light fluorescence from the particle.

A sixth aspect provides a method including administering a substance to the patient, binding the substance to the vulnerable plaque, activating a substance radiopaque characteristic upon the binding, and detecting the activated substance radiopaque characteristic.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 is a schematic view of a patient undergoing a vulnerable plaque detection procedure in accordance with the present invention;

FIG. 2 is a schematic view of a vulnerable plaque detection system including a detection device, in accordance with the present invention;

FIG. 3 is a block diagram summarizing three methods of vulnerable plaque detection, in accordance with the present invention;

FIG. 4 is a schematic view of a detection agent in accordance with the present invention; and

FIG. 5 is a schematic view of a detection agent particle in accordance with the present invention.

### DESCRIPTION OF THE PRESENTLY PREFERRED EMBODIMENTS

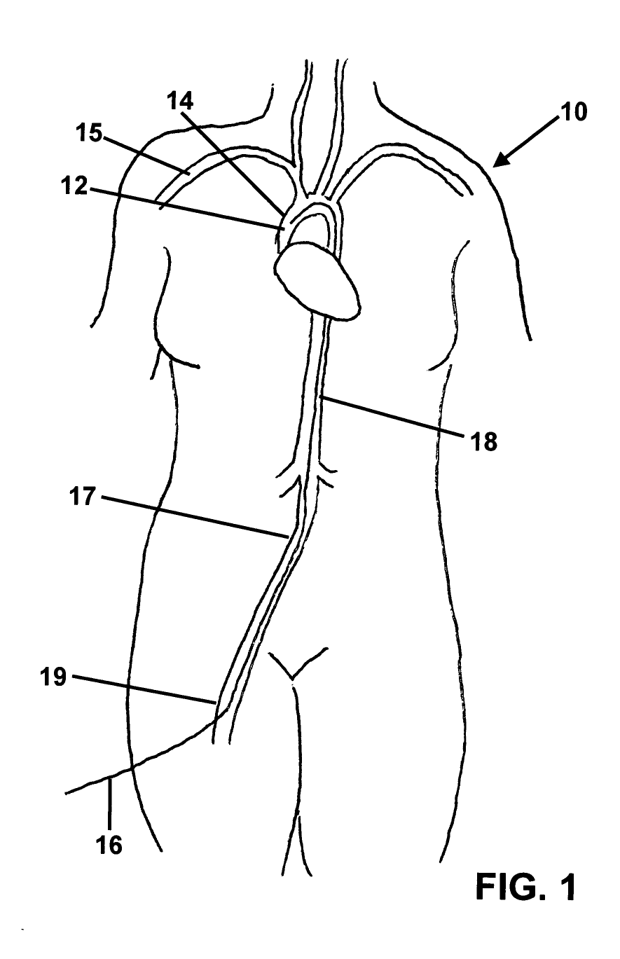

Referring to the drawings, wherein like reference numerals refer to like elements, FIG. 1 is a schematic view of a patient, indicated generally by numeral 10, undergoing a vulnerable plaque detection procedure in accordance with the present invention. A vulnerable plaque is distinguishable from other types of plaque, including hard plaques, by the presence of a fibrous cap. The vulnerable plaque fibrous cap retains a pool of lipids and other contents, which may be released into the blood vessel upon rupture. The released contents may form emboli that can lodge in a blood vessel thereby posing a risk to the patient. Vulnerable plaques, unlike hard plaques, are generally non-occlusive and as such, may not produce angina. The following description pertains to systems and methods for the detection of such vulnerable plaques.

Those skilled in the art will recognize that although the present invention is described primarily in the context of detecting vulnerable plaque while using specific diagnostic agents, substances, particles, and devices, the inventors contemplate broader systems and methods of application. Any number of agents, substances, particles, and devices capable of performing the prescribed function(s) may be compatible with the present invention. Furthermore, the detection of vulnerable plaque is not limited to the described strategies. Numerous modifications, substitutions, and variations may be made to the systems and methods while providing effective vulnerable plaque detection consistent with the present invention.

In the following description, vulnerable plaque detection is described primarily in the context of an endovascular catheterization detection procedure for a patient. The detected vulnerable plaque(s) may optionally be treated in the same or subsequent procedure. In either case, the patient may be treated in a clinical setting thereby allowing for controlled treatment in an environment in which immediate care is given. Treating the vulnerable plaque(s) during the same procedure as detection, however, may prevent the accidental or unanticipated release of emboli in a non-clinical setting. As such, complications stemming from vulnerable plaque rupture, such as heart attack and stroke, may be avoided. It should be noted that the terms “detect” and derivatives thereof, when used in regard to vulnerable plaque, refer to the diagnosis and localization of the lesion.

As shown in FIG. 1, patient 10 diagnostic site 12, which in this case is in an aortic vessel 14, may be accessed through various blood vessels. In one embodiment of the present invention, the diagnostic site 12 may be accessed percutaneously through an incision made in patient 10 femoral artery 19. In another embodiment, another vessel such as a subclavian artery 15 may be used to access the diagnostic site 12. Endovascular devices 20 such as guidewires, detectors, catheters, and the like for detection and/or treatment of the vulnerable plaque may be advanced to the diagnostic site 12 through a vessel pathway, which in this case includes an iliac artery 17 and abdominal aorta 18. It is important to note that pathways and treatment site other than the ones described may be used with the present invention. For example, the coronary and/or carotid arteries may be investigated for the presence of vulnerable plaque with an appropriate access pathway chosen for the detection and treatment devices.

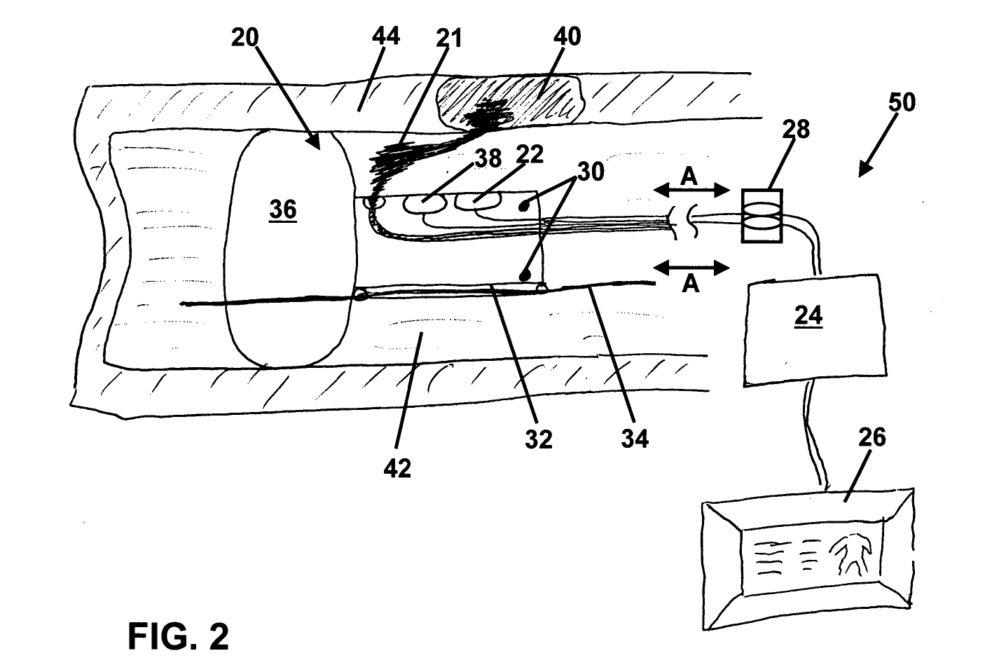

Referring now to FIG. 2, a schematic view of a vulnerable plaque detection system 50 including a detection device 20, in accordance with the present invention is shown. As described later, the device 20 is intended to function in concert with a detection agent 21 administered to the patient to facilitate vulnerable plaque 40 detection. A portion of the device 20 may be positioned within blood vessel lumen 42 and moved in an axial direction (e.g., shown by arrows A) thereby allowing diagnosis of a length of blood vessel 44. In one embodiment, the device 20 may include a signal-producing sensor 22 and a processor 24 that receives the signal outputted from the sensor 22. Sensor 22 may sense electromagnetic radiation including, but not limited to, radio wave radiation, microwave radiation, infrared radiation, visible light radiation, ultraviolet radiation, x-ray radiation, alpha radiation, beta radiation, gamma radiation, and fluorescence radiation. Processor 24 may perform analysis and determinations (e.g., calculations), including those based on equations or value tables, and may be a device such as a computer microprocessor.

Processor 24 may output information based on the analysis and determinations to an output device 26, such a video display. An operator (not shown), such as a physician, may monitor the progress of the detection procedure via the output device 26. Device 20 may include a movement control 28, such as a motorized pull-back device known in the art, to facilitate controlled device 20 axial movements. The processor 24 and/or the operator may control the movement control 28 and thus the device 20 movements.

Device 20 may include one or more, in this case two, radiopaque markers 30 to allow the operator to monitor the position of the device 20 within the blood vessel 44. Device 20 position may be determined by visualization methods known in the art, such as intravascular ultrasound (IVUS) and/or fluoroscopy. A guidewire sleeve 32 may be provided to slidably attach the device 20 to a guidewire 34. As is known in the art, the guidewire 34 provides a track upon which the device 20 travels through the involved vessel to the diagnostic site. Device 20 may include a lumen 35 formed therein for the administration of the detection agent 21 and/or therapeutic agents to the diagnostic site.

An expandable member 36, such as a balloon, may be operably attached to the device 20 to perform one or more functions. The uses of expandable members to perform various functions are known in the art. In one embodiment, the expandable member 36 may be used to deploy a mechanical vessel support or shunt, such as a stent or graft. In another or the same embodiment, the expandable member 36 may be used to create a temporary blood-less field at the diagnostic site. This may facilitate the delivery of the detection agent 21 and/or therapeutic agents by minimizing dilution and flow-away brought about by an intact bloodstream.

Device 20 may optionally include an emitter 38 for emitting electromagnetic radiation. Such emitters are known in the art and are typically used for endovascular imaging and diagnostic applications. In one embodiment, the emitter 38 may emit infra-red or near-infra-red radiation at a wavelength of about 700 to 3,000 nanometers, such as that produced by an optical coherence tomography (OCT) device. An example of an OCT imaging device, called an optical coherence domain reflectometer (OCDR), is disclosed in U.S. Pat. No. 5,321,501 issued to Swanson. The OCDR is capable of electronically performing two- and three-dimensional image scans over an extended longitudinal or depth range with sharp focus and high resolution and sensitivity over the range.

In another embodiment, the emitter 38 may emit another wavelength or range of wavelengths that may be used for the detection of the vulnerable plaque 40, such as those used in intravascular ultrasonic imaging systems or fluoroscopic systems. In yet another embodiment, the sensor 22 may detect other types of electromagnetic radiation produced by the detection agent 21, such as x-ray radiation, alpha radiation, beta radiation, or gamma radiation produced by a radioactive detection agent.

Those skilled in the art will recognize that a myriad of devices may be adapted for use with the present invention and the device 20 is demonstrative of merely one such possibility. Furthermore, various features of the device 20 may be omitted, substituted, re-arranged, re-configured, or added depending on the specifications of the vulnerable plaque detection procedure. Among other factors, the specifications of the procedure depend upon the nature of the vulnerable plaque detection agent 21 administered to the patient. The function of several of these agents will now be described.

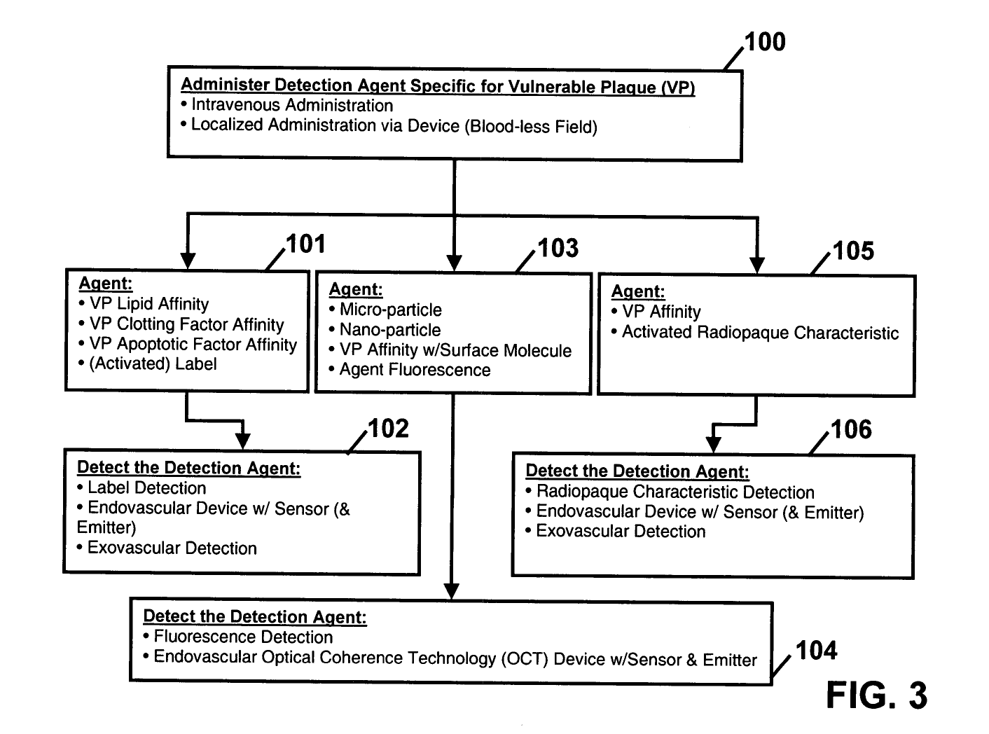

In a first possible mode of vulnerable plaque detection, as summarized in FIG. 3, the patient may be administered a detection agent (block 100) having affinity for at least one of a lipid, a clotting factor, or an apoptotic factor associated with the vulnerable plaque (block 101). The detection agent may be a biocompatible solution (e.g., a sterile saline based solution) administered intravenously prior to the detection procedure. After a selected amount of time (for example, 2 to 4 hours, although this time may vary greatly), the guide wire may be placed into the patient's body through a suitable insertion point. The guide wire is typically inserted to a length that is adequate to provide a guide for the device to the site of diagnosis and, optionally, treatment. The device is then inserted and follows along the guide wire to the diagnostic site. The device may then be slowly withdrawn axially while concurrently receiving sensor input. As such, vulnerable plaque that is labeled with the detection agent may be detected with the device (block 102).

Prior to and/or while the device is being withdrawn, the detection agent may be optionally administered via the device lumen. This provides an alternative or adjunct to intravenous administration. A blood-less field may also be optionally created via the expandable member or by other means to enhance detection agent administration.

Those skilled in the art will recognize that the strategy for detection agent administration may vary and is not limited to the examples provided. Numerous methods and devices for the detection of vulnerable plaque may be adapted for use with the present invention. By way of example, the detection step need not be performed with an endovascular device. In one embodiment, the vulnerable plaque may be detected from external the blood vessel (e.g., exovascularly). For example, a device for detecting the vulnerable plaque may be positioned through an incision in the patient. The device may then detect the vulnerable plaque without the need for catheterization. During such a procedure, detection may be achieved during open surgery or in a minimally invasive manner. As another example, the vulnerable plaque may be detected external to the patient, such as with an imaging device (e.g., magnetic resonance, ultrasound, or x-ray). Such procedures are sometimes referred to as whole body or cardiovascular scans.

The detection agent may have affinity for a lipid associated with the vulnerable plaque. As such, the detection agent specifically associates with and accumulates with regions of the vulnerable plaque, which include lipid-rich pools. In one embodiment, the detection agent may associate with a lipid molecule including at least one non-polar moiety. In another or the same embodiment, the detection agent may associate with a lipid molecule such as an apolipoprotein, saturated lipid, (poly-)unsaturated lipid, triglyceride, trans-fatty acid, cholesterol, and the like known to be present in or associated with vulnerable plaque.

The detection agent may have affinity for a clotting factor associated with the vulnerable plaque. One or more clotting factors may be associated with the vulnerable plaque either prior to or after its rupture. In one embodiment, the detection agent may associate with a clotting factor molecule such as a platelet associated molecule, fibrin, fibrinogen, prothrombin, thrombin, plasmin, plasminogen, serotonin, thromboxane A2, a kallikrein, thromboplastin, calcium ion, proaccelerin, proconvertin, antihemophilic factor, plasma thromboplastin component, Stuart-Prower factor, plasma thromboplastin antecedent, Hageman factor, fibrin stabilizing factor, and the like known to be present in or associated with vulnerable plaque

The detection agent may have affinity for an apoptotic factor associated with the vulnerable plaque. Vulnerable plaques are known to include areas of active cell apoptosis and therefore contain numerous apoptotic factors. A great deal of research has focused on apoptosis, apoptotic factors, and strategies for detection: For example, see “Cytochemical Methods for the Detection of Apoptosis” by M. Willingham (Journal of Histochemistry and Cytochemistry; vol. 47(9);1101-1109, 1999) and “Molecules Involved in Cell Death and Peripheral Tolerance” by J. Wang and M. J. Lenardo (Current Opinion in Immunology; vol. 9;818-825, 1997). In one embodiment, the detection agent may associate with an apoptotic factor molecule such as a cytokine, growth factor, caspase, serine-threonine protein kinase, phosphatidylinositol 3-kinase, protein kinase B, cytochrome c, NF-_{κ}B, forkhead, Bcl-2, Bcl-2-associated death promoter (BAD), Bcl-x_{L}, annexin, Fas ligand, tumor necrosis factor, and the like known to be present in or associated with vulnerable plaque

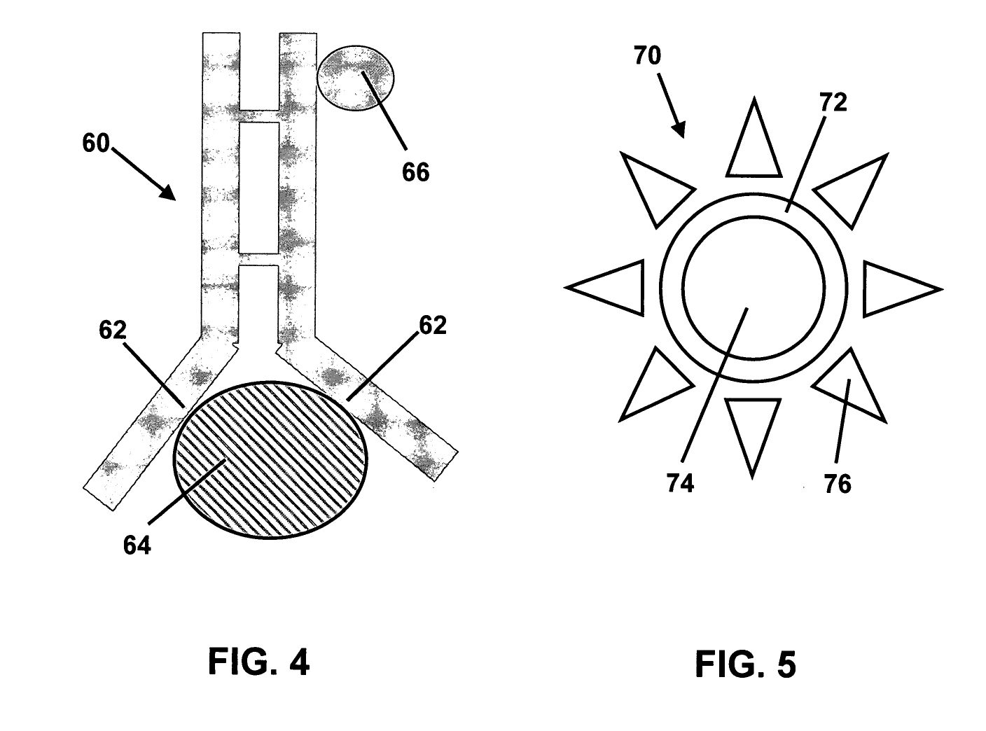

FIG. 4 is a schematic view of a detection agent 60 including one or more, in this case two, binding regions 62 that associates with a target molecule 64. The detection agent 60 may be, for example, an antibody targeted against a target molecule 64 or epitope thereof of a lipid, clotting factor, or apoptotic factor. As such, the detection agent 60 may associate with and/or accumulate within the vulnerable plaque. To facilitate detection, the detection agent 60 may include a label 66 such as a radioactive label, a fluorescent label, a radiopaque label, a paramagnetic label, a detectable heavy element, a detectable rare earth ion, and the like. The label 66 may be adapted to be easily detected by the device sensor. The label 66 may further be activated upon association of the detection agent 60 with the vulnerable plaque. For example, the molecular structure of the label 66 may change upon binding of the binding regions 62 to the target molecule 64 thereby allowing the detection agent 60 to be detected only while associated with the vulnerable plaque. Those skilled in the art will recognize that the structure and number of the binding region(s) 62, target molecule(s) 64, and label(s) 66 may vary while still providing effective detection of the vulnerable plaque.

In a second possible mode of the invention illustrated in FIG. 3, the patient may be administered a detection agent (block 100) of at least one type of particle (block 103). In one embodiment, the detection agent may be a micro-particle of about 0.5 to 10.0 micrometers in diameter. As shown in FIG. 5, a detection agent 70, such as a micro-particle shaped as a micro-sphere commonly known in the art, may be comprised of a protein shell 72 filled with air/gas 74. The detection agent 70 may include a surface coating 76 with affinity for one or more components of the vulnerable plaque. In another embodiment, the detection agent may be a nano-particle of about 10 to 200 nanometers in diameter. The detection agent may be a polymer nano-composite, a nano-powder, and/or a nano-tube of the fullerene family of carbon molecules as known in the art. Such detection agents may be manufactured from metals, alloys, polymers, or organic materials and may include a surface coating with affinity for one or more components of the vulnerable plaque.

The detection agent surface coating may include a surface molecule such as C-reactive protein binding molecule (CRPBP) that binds to C-reactive protein (CRP), which is released by tissue in response to acute injury, infection, or other inflammatory stimuli. Vulnerable plaques typically contain inflammatory cells that secret CRP thereby providing a specific target for the particle. The NycoCard® kit by Axis Shield includes a CRBP antibody that may be used as the particle surface coating of the present invention.

The detection agent is adapted to enhance the detection of the vulnerable plaque by the device. Detection of the detection agent and thus the vulnerable plaque may be achieved when the particle absorbs one wavelength of light and emits light radiation (e.g., fluorescence) of a different wavelength (block 104). The device shown in FIG. 2 may be an OCT device whereby the emitter illuminates the particle with infra-red and/or near infra-red radiation and the sensor detect the resulting fluorescence. The particle may be differentially sized to provide a unique light fluorescence wavelength. As such, various sized particles may be used in a single diagnostic “cocktail” to differentially label components of the vulnerable plaque.

In a third possible mode of the invention illustrated in FIG. 3, the patient may be administered a detection agent (block 100) that binds to the vulnerable plaque (block 105). The detection agent includes a radiopaque characteristic that activates upon the binding. In one embodiment, the radiopaque characteristic may be a molecular functional group included in the detection agent that is opaque to incident X-rays. The radiopaque characteristic is selectively activated typically at a predetermined temperature, pH, or other condition specific to the vulnerable plaque. The vulnerable plaque typically exhibits conditions such as elevated temperature that may be used to selectively activate the radiopaque characteristic. The radiopaque characteristic may therefore be activated within or adjacent to the vulnerable plaque thereby allowing it to discriminated from surrounding healthy tissue. In one embodiment, the detection agent may be an antibody including several heavy metal atoms comprising the radiopaque characteristic

After the radiopaque characteristic is selectively activated, the detection agent may then be detected via the endoscopic device or other imaging means (block 106). X-ray or magnetic resonance imaging (MRI) may be used to detect the activated radiopaque characteristic and thus the presence and location of vulnerable plaque within the patient. This provides a non-invasive strategy for the detection of vulnerable plaque. Those skilled in the art will recognize that a myriad of strategies exist for the detection of the activated radiopaque characteristic and that those strategies may be adapted for use with the present invention.

While the embodiments of the invention disclosed herein are presently considered to be preferred, various changes and modifications may be made without departing from the spirit and scope of the invention. The systems and methods of the present invention are not limited to any particular design, configuration, or sequence. Specifically, the detection procedure step order and devices for achieving the same may vary without limiting the utility of the invention. For example, the detection agent may be detected endoscopically with a variety of devices or by imaging means external to the patient and/or blood vessel. The functions ascribed to the aforementioned devices may be achieved with a single or with multiple devices. Furthermore, the detected vulnerable plaque may be treated concomitantly to the detection process or in a later procedure.

Upon reading the specification and reviewing the drawings hereof, it will become immediately obvious to those skilled in the art that myriad other embodiments of the present invention are possible, and that such embodiments are contemplated and fall within the scope of the presently claimed invention. The scope of the invention is indicated in the appended claims, and all changes that come within the meaning and range of equivalents are intended to be embraced therein.

1. A system for detecting a vulnerable plaque associated with a blood vessel of a patient, the system comprising:
a substance that is administered to the patient, the substance having affinity for at least one of a lipid, a clotting factor, or an apoptotic factor associated with the vulnerable plaque; and
a device that detects the substance.

2. The system of claim 1 wherein the substance comprises a label selected from a group consisting of a radioactive label, a fluorescent label, a radiopaque label, a paramagnetic label, a detectable heavy element, or a detectable rare earth ion.

3. The system of claim 2 wherein the label is activated upon association of the substance with the vulnerable plaque.

4. The system of claim 1 wherein the lipid comprises a molecule including at least one non-polar moiety.

5. The system of claim 1 wherein the lipid comprises a molecule selected from a group consisting of apolipoproteins, saturated lipids, (poly-)unsaturated lipids, triglycerides, trans-fatty acids, and cholesterol.

6. The system of claim 1 wherein the clotting factor comprises a molecule selected from a group consisting of a platelet associated molecule, fibrin, fibrinogen, prothrombin, thrombin, plasmin, plasminogen, serotonin, thromboxane A2, a kallikrein, thromboplastin, calcium ion, proaccelerin, proconvertin, antihemophilic factor, plasma thromboplastin component, Stuart-Prower factor, plasma thromboplastin antecedent, Hageman factor, and fibrin stabilizing factor.

7. The system of claim 1 wherein the apoptotic factor comprises a molecule selected from a group consisting of cytokines, growth factors, caspases, serine-threonine protein kinases, phosphatidylinositol 3-kinase, protein kinase B, cytochrome c, NF-_{κ}B, forkhead, Bcl-2, Bcl-2-associated death promoter (BAD), Bcl-x_{L}, annexins, Fas ligands, and tumor necrosis factor.

8. The system of claim 1 wherein the device comprises an endovascular device.

9. A system for detecting a vulnerable plaque associated with a blood vessel of a patient, the system comprising:
a particle that is administered to the patient;
an emitter that emits infra-red or near infra-red radiation on the particle; and
a detector that detects light fluorescence from the particle.

10. The system of claim 9 wherein the particle comprises a micro-particle of about 0.5 to 10.0 micrometers in diameter.

11. The system of claim 9 wherein the particle comprises a nano-particle of about 10 to 200 nanometers in diameter.

12. The system of claim 9 wherein the particle is sized to provide a unique light fluorescence wavelength.

13. The system of claim 9 wherein the particle comprises a surface molecule with affinity for the vulnerable plaque.

14. The system of claim 13 wherein the surface molecule comprises a C-reactive protein binding molecule.

15. The system of claim 9 wherein the emitter and detector comprise an optical coherence tomography device.

16. A system for detecting a vulnerable plaque associated with a blood vessel of a patient, the system comprising:
a substance that is administered to the patient, the substance having affinity for the vulnerable plaque and including a substance radiopaque characteristic that activates upon association of substance with the vulnerable plaque; and
a device that detects the substance radiopaque characteristic.

17. The system of claim 16 wherein the substance radiopaque characteristic is activated at a predetermined temperature.

18. The system of claim 16 wherein the substance radiopaque characteristic is activated at a predetermined pH.

19. The system of claim 16 wherein the device comprises an endovascular device.

20. A method of detecting a vulnerable plaque associated with a blood vessel of a patient, the method comprising:
administering a substance to the patient;
binding the substance to at least one of a lipid, a platelet, or an apoptotic factor associated with the vulnerable plaque; and
detecting the bound substance.

21. The method of claim 20 wherein the substance comprises a label selected from a group consisting of a radioactive label, a fluorescent label, a radiopaque label, a paramagnetic label, a detectable heavy element, or a detectable rare earth ion.

22. The method of claim 21 wherein the label is activated upon the binding of the substance.

23. The method of claim 20 wherein the lipid comprises a molecule including at least one non-polar moiety.

24. The method of claim 20 wherein the lipid comprises a molecule selected from a group consisting of an apolipoprotein, a saturated lipid, a (poly-)unsaturated lipid, a triglyceride, a trans-fatty acid, and cholesterol.

25. The method of claim 20 wherein the clotting factor comprises a molecule selected from a group consisting of a platelet associated molecule, fibrin, fibrinogen, prothrombin, thrombin, plasmin, plasminogen, serotonin, thromboxane A2, a kallikrein, thromboplastin, calcium ion, proaccelerin, proconvertin, antihemophilic factor, plasma thromboplastin component, Stuart-Prower factor, plasma thromboplastin antecedent, Hageman factor, and fibrin stabilizing factor.

26. The method of claim 20 wherein the apoptotic factor comprises a molecule selected from a group consisting of a cytokine, a growth factor, a caspase, a serine-threonine protein kinase, phosphatidylinositol 3-kinase, protein kinase B, cytochrome c, NF-_{κ}B, forkhead, Bcl-2, Bcl-2-associated death promoter (BAD), Bcl-x_{L}, an annexin, Fas ligand, and tumor necrosis factor.

27. The method of claim 20 wherein the device comprises an endovascular device.

28. A method of detecting a vulnerable plaque associated with a blood vessel of a patient, the method comprising:
administering a particle to the patient;
emitting infra-red or near infra-red radiation on the particle; and
detecting light fluorescence from the particle.

29. The method of claim 28 wherein the particle comprises a micro-particle of about 0.5 to 10.0 micrometers in diameter.

30. The method of claim 28 wherein the particle comprises a nano-particle of about 10 to 200 nanometers in diameter.

31. The method of claim 28 wherein the particle is sized to provide a unique light fluorescence wavelength.

32. The method of claim 28 wherein the particle comprises a surface molecule with affinity for the vulnerable plaque.

33. The method of claim 32 wherein the surface molecule comprises a C-reactive protein binding molecule.

34. The method of claim 28 wherein the emitter and detector comprise an optical coherence tomography device.

35. A method of detecting a vulnerable plaque associated with a blood vessel of a patient, the system comprising:
administering a substance to the patient;
binding the substance to the vulnerable plaque;
activating a substance radiopaque characteristic upon the binding; and
detecting the activated substance radiopaque characteristic.

36. The method of claim 35 wherein the substance radiopaque characteristic is activated at a predetermined temperature.

37. The method of claim 35 wherein the substance radiopaque characteristic is activated at a predetermined pH.
